(19) United States
(12) Patent Application Publication
(10) Pub. No.: US 2023/0364382 A1
(43) Pub. Date: Nov. 16, 2023

(54) CATHETER SHAFT WITH TAPERED INNER DIAMETER FOR IMPROVED FLOW RATE

(71) Applicant: Canon U.S.A., Inc., Melville, NY (US)

(72) Inventor: Benedict Shia, Cambridge, MA (US)

(21) Appl. No.: 18/018,976

(22) PCT Filed: Aug. 6, 2021
(86) PCT No.: PCT/US21/45052

§ 371 Date: Jan. 31, 2023

Related U.S. Application Data
(60) Provisional application No. 63/062,208, filed on Aug. 6, 2020.

(51) Int. Cl. A61M 25/00

(52) U.S. Cl. CPC A61M 25/0028; A61M 25/0053

(57) ABSTRACT

The subject apparatus and methods include a bendable catheter having a tapered body allowing for advancement of the catheter deeper into the anatomy, while retaining adequate fluid flow through the catheter.

### CROSS REFERENCE TO RELATED PATENT APPLICATIONS

This application claims priority from U.S. Provisional Patent Application No. 63/062,208, filed on Aug. 6, 2020, in the United States Patent and Trademark Office, the disclosure of which is incorporated herein in its entirety by reference.

### FIELD OF THE DISCLOSURE

The present disclosure relates generally to apparatus and methods for medical device. More particularly, the subject disclosure is directed to apparatus and methods for a snake catheter for use in medical applications, capable of achieving a decreased outer diameter while retaining optimal fluid flow rates.

### BACKGROUND OF THE DISCLOSURE

Currently endoscopic procedures are performed with irrigation and suction applied through the endoscope's working channel. There is a camera permanently integrated into the distal tip of the endoscope, so it is external to the working channel of the endoscope, leaving the full diameter of the working channel to allow suction/irrigation.

US 2019/0105468 A1: “Medical continuum robot with multiple bending sections”. This is directed to an articulated medical device having a hollow cavity, wherein the device is capable of maneuvering within a patient, and allowing a medical tool to be guided through the hollow cavity for medical procedures, including endoscopes, cameras, and catheters.

U.S. RE46007E1: “Automated control of irrigation and aspiration in a single use endoscope”. This relates to an integrated and automated irrigation and aspiration system for use in an endoscopic imaging system.

U.S. Pat. No. 5,549,547A: Flexible tube having a tapered diameter portion for use with endoscopic irrigation instruments relates to an endoscopic irrigation instrument includes a fluid chamber, a cannula coupled to a distal end of the fluid chamber, a tapered irrigation tube which couples at its proximal end to a fluid source and which couples at its distal end to an irrigation port of the fluid chamber, and a pinch valve which controls the flow of fluid from the irrigation source to the fluid chamber and hence through the cannula to the surgical site.

U.S. Pat. No. 8,517,999B2: Irrigated catheter with improved fluid flow relates to an irrigated catheter with uniform cooling and/or uniform fluid distribution in longitudinally spaced apart elution holes by varying the diameter of a fluid delivery lumen.

During endoscopic procedures, physicians will irrigate and suction through the working channel of the endoscope. For example, As shown in FIG. 1 (prior art), the Olympus BF-H190 endoscope has a 5.5 mm outside diameter (“OD”) with a 2.0 mm inside diameter (“ID”) working channel.

The present Snake Robotic Catheter 10, shown in FIG. 2, incorporates an impressive 3.7 mm OD 12 with a 2.5 mm ID multilumen extrusion 14, as the main component that makes up the catheter shaft 16 and most of its working length. At the distal end 18, a 2.2 mm ID single lumen extrusion (Inner Cover) 20 is bonded to the ID of the multilumen extrusion 14, and is about 100 mm in length. Due to its smaller OD, the Snake Robotic Catheter 10 can reach smaller and more distal anatomy than a typical endoscope.

However, the camera 22 providing visualization during procedures for the Snake Robotic Catheter 10 is 1.6 mm in diameter, and occupies the center lumen 24. The gap between the OD of the camera 22 and the ID of the Catheter 10 has a smaller cross sectional area than the working channel of an endoscope; therefore, fluid flow through of the Snake Robotic Catheter 10 will be less than that through the endoscope working channel. The present disclosure provides a solution to this issue.

### SUMMARY

Thus, to address such exemplary needs in the industry, the presently disclosed device teaches a multi-section catheter comprising: a bendable section having an outer diameter; and a proximal section, having an outer diameter, attached to the bendable section, wherein a transition from the bendable section to the proximal section is tapered, such that the outer diameters from the bendable section to the proximal section increases in the taper.

In other embodiments, the outer diameter of the bendable section of the catheter is less than the outer diameter of the proximal section.

In further embodiment, the inner diameter of the proximal section is configured to accommodate at least two tools. Furthermore, the at least two tools may include a camera, needle, probe and other medical devices.

In additional embodiments, the catheter can perform endoscopic-type procedures, but will be able to navigate to more distal anatomy, and achieve a smaller bend radii due to a smaller bending section profile.

Further embodiments demonstrate that the catheter has a tapered inner diameter in the proximal section which provides an increase of fluid flow rates in the catheter.

It is further contemplated that the catheter comprises a braided tube abutting the bending section. Furthermore, the braided tube may be a progressive braid.

In additional embodiments the proximal section of the catheter is stiffer than the bending section, concentrating the bending at the bending section and providing more pushability of the catheter.

The subject innovation further teaches a method for using a multi-section catheter comprising: providing a multi-section catheter having: a bendable section; and a proximal section attached to the bendable section, wherein a transition from the bendable section to the proximal section is tapered, followed by inserting the catheter into a subject to a desired target using a camera; and flowing fluid through the catheter to the target.

It is further contemplated in additional embodiments that the transition from the bendable section to the proximal section is tapered such that the outer diameters from the bendable section to the proximal section increases in the taper.

In other embodiments, the outer diameter of the bendable section of the catheter is less than the outer diameter of the proximal section.

In further embodiment, the inner diameter of the proximal section is configured to accommodate at least two tools. Furthermore, the at least two tools may include a camera, needle, probe and other medical devices.

In additional embodiments, the catheter can perform endoscopic-type procedures, but will be able to navigate to more distal anatomy, and achieve a smaller bend radii due to a smaller bending section profile.

Further embodiments demonstrate that the catheter has a tapered inner diameter in the proximal section which provides an increase of fluid flow rates in the catheter.

It is further contemplated that the catheter comprises a braided tube abutting the bending section. Furthermore, the braided tube may be a progressive braid.

In additional embodiments the proximal section of the catheter is stiffer than the bending section, concentrating the bending at the bending section and providing more pushability of the catheter.

These and other objects, features, and advantages of the present disclosure will become apparent upon reading the following detailed description of exemplary embodiments of the present disclosure, when taken in conjunction with the appended drawing guidance device 10, and provided paragraphs.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIG. 1 is a specification view of a prior art medical device (endoscope) showing the existing limitations in the existing prior art.

FIG. 2 depicts a side perspective view of an exemplary medical device in the existing prior art.

FIG. 3 provides a side perspective view of an exemplary medical device according to one or more embodiment of the subject innovation.

FIG. 4 also depicts a side perspective view of an exemplary medical device according to one or more embodiment of the subject innovation.

FIG. 5 depicts a side perspective view of an exemplary medical device according to one or more embodiment of the subject innovation.

FIG. 6 is a chart depicting various flow rates for the subject innovation, as well as comparative flow rates for alternative embodiments and samples.

FIG. 7 shows a close-up side perspective view of an exemplary medical device according to one or more embodiment of the subject innovation.

FIG. 8 depicts a close-up side perspective view of an exemplary medical device according to one or more embodiment of the subject innovation.

FIG. 9 provides a side perspective view of an exemplary medical device according to one or more embodiment of the subject innovation.

FIG. 10 depicts a side perspective view of an exemplary medical device according to one or more embodiment of the subject innovation.

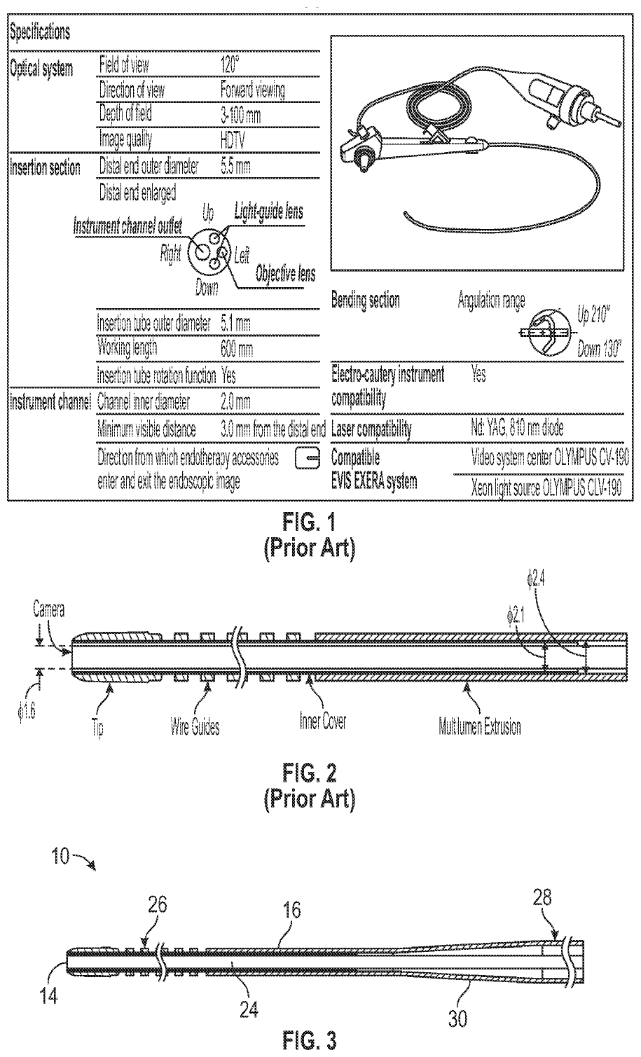

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

In referring to the description, specific details are set forth in order to provide a thorough understanding of the examples disclosed. In other instances, well-known methods, procedures, components and materials have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description and/or illustration to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings guidance device 10 herein.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated. The term “position” or “positioning” should be understood as including both spatial position and angular orientation.

As detailed in FIG. 3, the present disclosure details a Snake Robotic Catheter (“catheter”) 10 which has a steerable device with a distal bending section 26. The bending section 26 is designed to bend to a tight radius, and is sized approximately with a 3.3 mm outside diameter 12 in order to access deeper, smaller anatomy. The proximal section 28 of the Snake Robotic Catheter 10 does not need to bend to the same radius as the bending section 26, and is not intended to reach deep into the anatomy, so the outer diameter of the proximal section 28 can be larger, (e.g. the same OD as an endoscope). The multilumen extrusion 14 can be extruded such that it tapers 30 up in OD and ID (known in the extrusion industry as a bump extrusion). The distal bending section 26 will remain at approximately 3.3 mm OD to allow passage into smaller anatomy, but the proximal section 28 can be bumped up to a larger OD (equivalent to current endoscopes) and ID. This larger ID of the proximal section 28 will increase flow rate through the catheter with the camera 22 remaining at the distal end 18 of the bending section 26.

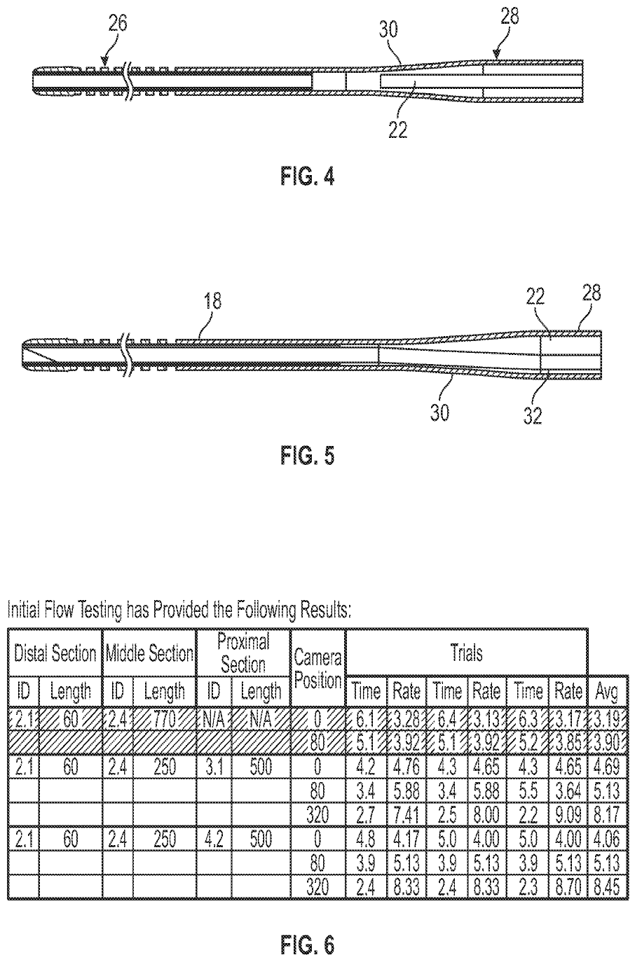

If greater flow is desired, the camera 22 can be retracted back out of the distal end 18 of bending section 26, into the portion where the ID is larger, resulting in even better flow. This retracted position may be seen in FIG. 4, showing the additional space needed for increased fluid flow rate.

FIG. 5 further details an increase in the taper 30, which can potentially increase the ID at the proximal section 28 enough so that an additional instrument 32 (here a biopsy needle) can be inserted into the catheter 10, up to the beginning of the taper 30. This would allow a more rapid exchange between the camera 30 and instrument 32 since neither would have to be completely removed from the catheter 10 in order to be used.

Flow testing results are provided in FIG. 6, which shows optimal flow rates using the subject tapered catheter 10. As can be seen in the results, the increased flow rates achieved far exceed those of the existing art, and advance the technology while allowing for deeper and more precise insertion of the catheter 10 into the anatomy.

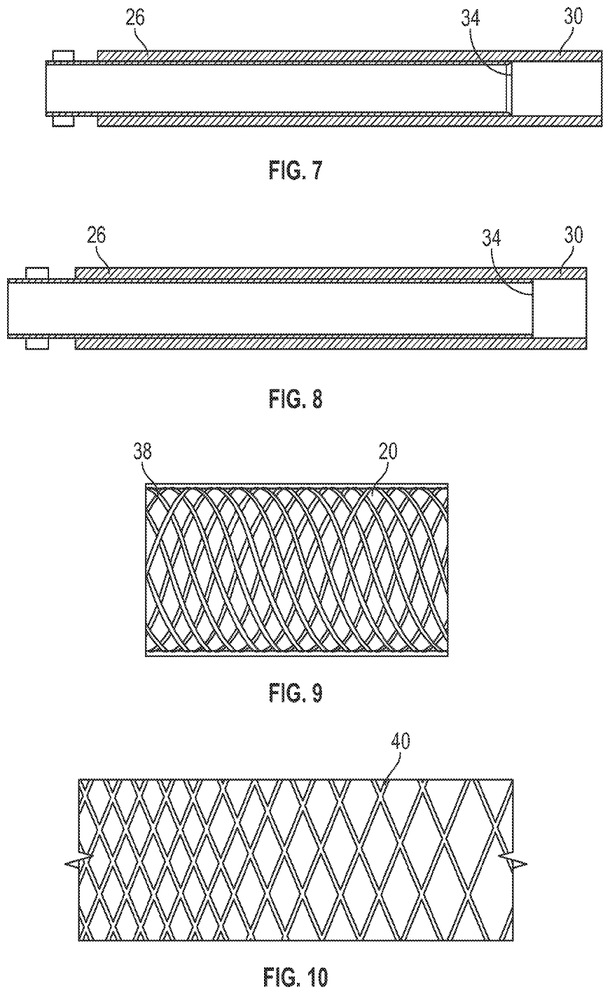

FIGS. 7 and 8 provide additional embodiment of the subject catheter 10, wherein the transition from the tapered 30 end to the bending section 26 is further detailed. FIG. 7 provides a gradual edge 34, showing a transition at approximately 45 degrees, whereas FIG. 8 shows a stepped edge 36 transition point. The transition are incorporated to improve fluid flow and reduce possible interference with the camera 22 or other instrument 32.

FIG. 9 introduces the use of a braided structure 38 for reinforcing the inner cover 20 of the catheter 10. The braided structure 38 may be incorporated into the inner cover or positioned between the inner cover 20 and multilumen extrusion 14. The structure of the braids allows for bending without adding additional stress, while increasing wall strength. As can be appreciated, tighter or looser braids may be incorporated to achieve optimal strength and/or rigidity in the catheter 10.

As seen in FIG. 10, a progressive braid structure 40 may be incorporated for further refinement of the catheter 10. In yet another embodiment, the braid structure may be rounded at either or both ends to ease in transitions and movement of the camera 22 or instrument 32 through the catheter 10.

Exemplary features therefore include: multi-section, steerable distal section, stiffer proximal section, camera in center lumen, camera positioned at distal tip and at taper, cameral selectively secured at proximal end, allows irrigation and/or suction, tapered ID, tapered OD, taper location and tapered ID accommodates multiple instruments.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A multi-section catheter comprising:
a bendable section having an outer diameter; and
a proximal section, having an outer diameter, attached to the bendable section,
wherein a transition from the bendable section to the proximal section is tapered.

2. The catheter of claim 1, wherein the outer diameter of the bendable section is less than the outer diameter of the proximal section.

3. The catheter of claim 1, wherein an inner diameter of the proximal section is configured to accommodate at least two tools.

4. The catheter of claim 4, where the at least two tools are selected from the group comprising of a camera, needle, probe and other medical devices.

5. The catheter of claim 1, wherein the catheter can perform endoscopic-type procedures, but will be able to navigate to more distal anatomy, and achieve a smaller bend radii due to a smaller bending section profile.

6. The catheter of claim 1, wherein a tapered inner diameter of the proximal section provides an increase of fluid flow rates in the catheter.

7. The catheter of claim 1, further comprising a braided tube abutting the bending section.

8. The catheter of claim 7, wherein the braided tube is progressive.

9. The catheter of claim 1, wherein the proximal section is stiffer than the bending section, concentrating the bending at the bending section and providing more pushability of the catheter.

10. A method for using a multi-section catheter comprising:
providing a multi-section catheter having:
a bendable section; and
a proximal section attached to the bendable section,
wherein a transition from the bendable section to the proximal section is tapered,
inserting the catheter into a subject to a desired target using a camera; and
flowing fluid through the catheter to the target.

11. The method of claim 10, wherein an outer diameter of the bendable section is less than an outer diameter of the proximal section.

12. The method of claim 10, wherein the camera is retracted to the proximal section before flowing fluid through the catheter to the target.

13. The method of claim 10, wherein an inner diameter of the proximal section is configured to accommodate at least two tools.

14. The method of claim 13, where the at least two tools are selected from the group comprising of a camera, needle, probe and other medical devices.

15. The method of claim 10, wherein the catheter can perform endoscopic-type procedures, but will be able to navigate to more distal anatomy, and achieve a smaller bend radii due to a smaller bending section profile.

16. The method of claim 1, wherein a tapered inner diameter of the proximal section provides an increase of fluid flow rates in the catheter.

17. The method of claim 1, wherein the catheter further comprises a braided tube abutting the bending section.

18. The method of claim 17, wherein the braided tube is progressive.

19. The method of claim 1, wherein the proximal section is stiffer than the bending section, concentrating the bending at the bending section and providing more pushability of the catheter.
